(19) United States
(12) Patent Application Publication
(10) Pub. No.: US 2016/0278702 A1
(43) Pub. Date: Sep. 29, 2016

(54) PHYSIOLOGICAL SIGNAL MONITORING BELT

(71) Applicant: ZENTAN TECHNOLOGY CO., LTD., New Taipei City (TW)

(72) Inventor: TONG-PIE CHEN, NEW TAIPEI CITY (TW)

(21) Appl. No.: 14/665,048

(22) Filed: Mar. 23, 2015

(51) Int. Cl. A61B 5/00; A61B 5/0476; A61B 5/0402; A61B 5/01; A61B 5/0245

(52) U.S. Cl. CPC A61B 5/6831; A61B 5/01; A61B 5/0245; A61B 5/0402; A61B 5/0476

(57) ABSTRACT

A physiological signal monitoring belt includes a belt-shaped unit and an electrode unit. The electrode unit is disposed on a surface of the belt-shaped unit and includes a base layer, an intermediate layer, and a conductive layer. The intermediate layer has a first surface and a second surface. The base layer is disposed on the first surface, and the conductive layer is disposed on the second surface. The electrode unit is disposed on the surface of the belt-shaped unit through the base layer.

### BACKGROUND OF THE INVENTION

1. Field of the Invention

The present disclosure relates to a physiological signal monitoring belt; in particular, to a physiological signal monitoring belt for monitoring physiological information of a living body.

2. Description of Related Art

With the advance of technology, medical equipment for monitoring various physiological information of the human body has been designed towards miniaturization and to have a wireless configuration. Therefore, physiological information of a person (especially a patient) can be monitored anytime, anywhere, and proper measurements can be made immediately according to the physiological information observed, reducing the number and the frequency of accessing health care institutions, and the disease can be effectively monitored to avoid unexpected situations. In addition, the physiological information observed can be used for evaluating the current physiological situation of an athlete when running.

In the market, a kind of physiological signal monitoring belt of the prior art, which can be tied around human thorax, utilizes the conductive fabric or conductive fibers disposed on the inner surface thereof that are able to be in touch with the skin of the human body, to monitor various physiological signals, and utilizes the wireless transmission device electrically connected thereto to transmit the physiological signals measured to a physiological signal measuring device for interpretation, analysis and display.

However, the conductive fabric or conductive fibers disposed on the inner surface of the physiological signal monitoring belt of the prior art has shortcomings such as poor measuring effect, poor measuring accuracy and noise. In other words, there are still inadequacies in using the physiological signal monitoring belt of the prior art, which need to be improved.

Therefore, how to provide a physiological signal monitoring belt that improves the monitoring effect and monitoring accuracy and overcomes the above mentioned shortcomings has become an important issue.

### SUMMARY OF THE INVENTION

The embodiment of the present disclosure provides a physiological signal monitoring belt utilizing a multi-layered composite electrode unit to improve the monitoring effect and increase the accuracy of the physiological signal detection thereof.

The embodiment of the present disclosure provides a physiological signal monitoring belt, which includes a belt-shaped unit and an electrode unit disposed on a surface of the belt-shaped unit. The electrode unit includes a base layer, an intermediate layer, and a conductive layer. The intermediate layer has a first surface and a second surface. The base layer is disposed on the first surface, and the conductive layer is disposed on the second surface. The electrode unit is disposed on the surface of the belt-shaped unit through the base layer.

The embodiment of the present disclosure further provides a physiological signal monitoring belt, which includes a belt-shaped unit, two electrode units, a first engaging element, and second engaging element. The belt-shaped unit has a first surface and a second surface. The two electrode units are disposed on the first surface of the belt-shaped unit and arranged corresponding to each other. The two electrode units each include a base layer, an intermediate layer, and a conductive layer. The intermediate layer has a first surface and a second surface. The base layer is disposed on the first surface of the intermediate layer, and the conductive layer is disposed on the second surface of the intermediate layer. The first engaging element is electrically connected to one of the two electrode units, and the second engaging element is electrically connected to the other of the two electrode units. Each of the two electrode units is disposed on the first surface of the belt-shaped unit through the corresponding base unit.

According to the present disclosure, the physiological signal monitoring belt can utilize a multi-layered composite electrode unit to improve the monitoring effect and increase the accuracy of the physiological signal detection thereof. Especially, the electrode unit utilizes a multi-layered structure that has a base layer, an intermediate layer, and a conductive layer to improve the signal monitoring accuracy of the physiological signal monitoring belt.

In order to further the understanding regarding the present disclosure, the following embodiments are provided along with illustrations to facilitate the disclosure of the present disclosure.

### BRIEF DESCRIPTION OF THE DRAWINGS

FIG. 1 shows an exploded view of a physiological signal monitoring belt according to a first embodiment of the present disclosure;

FIG. 2A shows a cross-sectional view of a configuration an electrode unit of the physiological signal monitoring belt according to the first embodiment and a second embodiment of the present disclosure;

FIG. 2B shows a cross-sectional view of another configuration the electrode unit of the physiological signal monitoring belt according to the first embodiment and the second embodiment of the present disclosure;

FIG. 3A shows an exploded view of the physiological signal monitoring belt according to the second embodiment of the present disclosure;

FIG. 3B shows a perspective view of the physiological signal monitoring belt according to the second embodiment of the present disclosure;

FIG. 3C shows a front view of the physiological signal monitoring belt according to the second embodiment of the present disclosure;

FIG. 4 shows a perspective view of the physiological signal monitoring belt in use according to the second embodiment of the present disclosure.

### DETAILED DESCRIPTION OF THE PREFERRED EMBODIMENTS

The aforementioned illustrations and following detailed descriptions are exemplary for the purpose of further explaining the scope of the present disclosure. Other objectives and advantages related to the present disclosure will be illustrated in the subsequent descriptions and appended drawings.

### First Embodiment

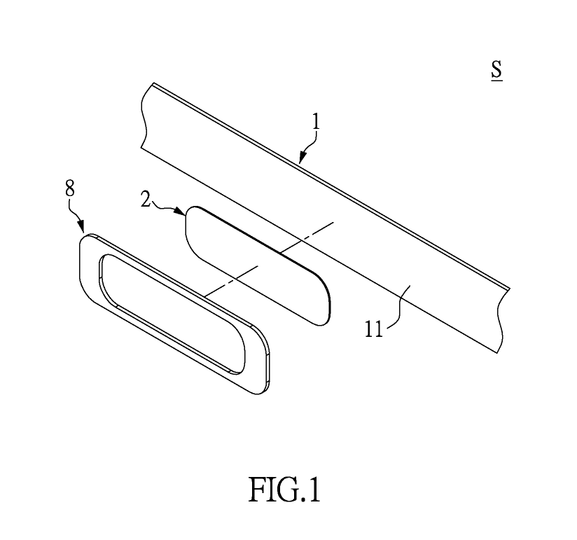

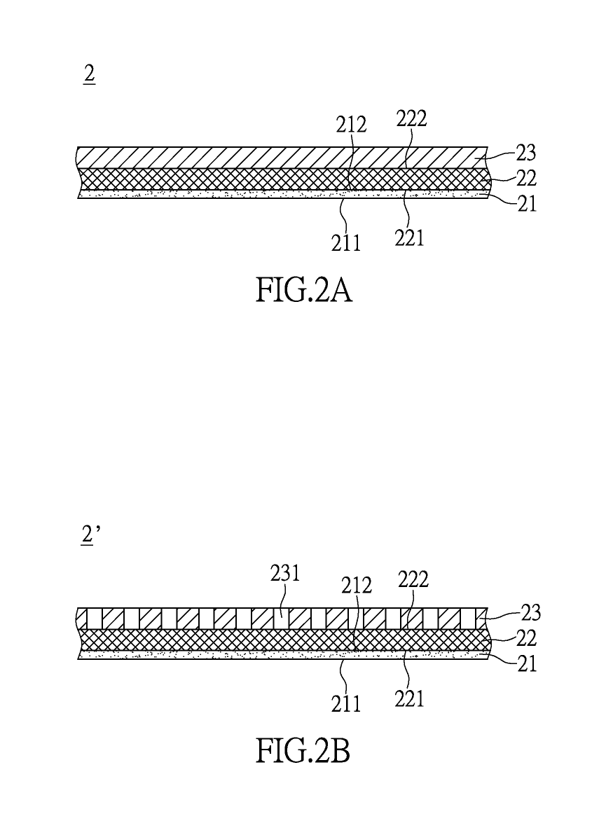

First, please refer to FIG. 1 to FIG. 2B. FIG. 1 shows an exploded view of a physiological signal monitoring belt according to a first embodiment of the present disclosure. FIG. 2A shows a cross-sectional view of a configuration an electrode unit of the physiological signal monitoring belt according to the first embodiment and a second embodiment of the present disclosure. FIG. 2B shows a cross-sectional view of another configuration of the electrode unit of the physiological signal monitoring belt according to the first embodiment and the second embodiment of the present disclosure. The first embodiment of the present disclosure provides a physiological signal monitoring belt S, which includes a belt-shaped unit 1 and an electrode unit 2. The physiological signal monitoring belt S is for monitoring physiological information, such as pulse, of a living body. In the first embodiment, the belt-shaped unit 1 has a surface 11, whereby the electrode unit 2 can be disposed on the surface 11 of the belt-shaped unit 1. For example, the electrode unit 2 can be attached to the belt-shaped unit 1 by hot pressing, coating, weaving, printing, or other molding process. Furthermore, the electrode unit 2 can be fixedly attached to the belt-shaped unit 1 through a layering unit 8 peripherally arranged on the electrode unit 2.

Please refer concurrently to FIG. 2A. Specifically, the electrode unit 2 may include a base layer 21, an intermediate layer 22, and a conductive layer 23. In other words, the electrode unit 2 can be formed with a multi-layered composite material. For example, the base layer 21, the intermediate layer 22, and the conductive layer 23 can be one-piece formed. Alternatively, the base layer 21, the intermediate layer 22, and the conductive layer 23 can be woven together to form a three-layered structure for forming the electrode unit 2. The intermediate layer 22 has a first surface 221 and a second surface 222. As a specific example, the first surface 221 and the second surface 222 can be the lower surface and the upper surface of the intermediate layer 22. The base layer 21 can be disposed on the first surface 221 of the intermediate layer 22, and the conductive layer 23 can be disposed on the second surface 222 of the intermediate layer 22. Therefore, the electrode unit 2 can be disposed on the surface 11 of the belt-shaped unit 1 through the base layer 21.

To put it concretely, in the first embodiment of the instant disclosure, the base layer 21 can be formed with an adhesive colloid layer. In addition, the base layer can be water-repellent and water-blocking. The base layer 21 has a first surface 211 and a second surface 212. As a specific example, the first surface 211 and the second surface 212 can be the lower surface and the upper surface of the base layer 21. The first surface 211 and the second surface 212 both can be adhesive. In an exemplary embodiment, merely one of the first surface 211 and second surface 212 is adhesive, but the present disclosure is not limited thereto. Since the base layer 21, the intermediate layer 22, and the conductive layer 23 can be one-piece formed for forming the electrode unit 2, the base layer 21 may not be adhesive. For example, the electrode unit 2 can be attached to the belt-shaped unit 1 by hot pressing, coating, weaving, printing, stitching, or other molding process.

Moreover, the intermediate layer 22 can be a water absorbent intermediate layer. For example, the intermediate layer may be formed with a water absorbent material, such as a water absorbent sponge, a water absorbent colloid, or a non-woven cloth, whereby the conductivity of the electrode unit 2 that is in touch with the skin of the living body can be enhanced by the water absorbed by the intermediate layer 22 that is penetrating the intermediate layer 22. In an alternative embodiment, the intermediate layer 22 can be made from natural fibers, man-made fibers, or the combination thereof, through a weaving process. The natural fibers can be any natural fiber, such as, but not limited to, cotton, hemp, silk or wool. The man-made fibers can be any man-made fibers, such as, but not limited to, rayon fibers, nylon fibers, polyester fibers or acrylic fibers. The woven fabrics for forming the intermediate layer 22 may be flat-woven, non-woven, mesh woven, and any other type, or the woven fabrics can be knitted fabrics.

Furthermore, the conductive layer 23 can be formed with a rubber conductive layer for monitoring the physiological information of the living body, and the present disclosure is not limited thereto. In an alternative embodiment, the conductive layer 23 may be formed with a conductive fabric, such as conductive cloth. Referring to FIG. 2B, the electrode unit 2′ can be formed with a plurality of micro pores 231 thereon. As a specific example, the micro pores 231 are disposed on the conductive layer 23. In the first embodiment of the present disclosure, the micro pores 231 formed on the conductive layer 23 are configured to penetrate the conductive layer 23, whereby the plurality of the micro pores 231 are exposing the second surface 222 of the intermediate layer 22, such that the water absorbed by the intermediate layer 22, which is water absorbent, can be brought sufficiently into the conductive layer 23 through the micro pores 231. Thus, the conductivity of the electrode unit 2′ that is in touch with the skin of the living body can be enhanced. In addition, the conductive layer 23 can be wettable to enhance the monitoring efficiency of the physiological signal monitoring belt S that is in touch with the skin of the living body, and the present disclosure is not limited thereto. Accordingly, the electrode unit 2, 2′ can be water-locking, thus the poor physiological signal monitoring effect can be avoided even when the electrode unit 2, 2′ is disposed in a dry environment.

In accordance with the first embodiment, the physiological signal monitoring belt S provided by the present disclosure utilizes the electrode unit 2 2′ that is formed with a multi-layered composite material to improve the monitoring effect and accuracy thereof. Specifically, the physiological signal monitoring belt S utilizes the electrode unit 2 2′ that includes the base layer 21, the intermediate layer 22, and the conductive layer 23, to improve the monitoring accuracy thereof Moreover, the physiological signal monitoring belt S utilizes the micro pores 231 disposed on the conductive layer 23 to transfer the moisture absorbed by the intermediate layer 22 to the conductive layer 23, thus to increase the conductivity of the electrode unit 2 2′ that is in touch with the skin of the living body.

### Second Embodiment

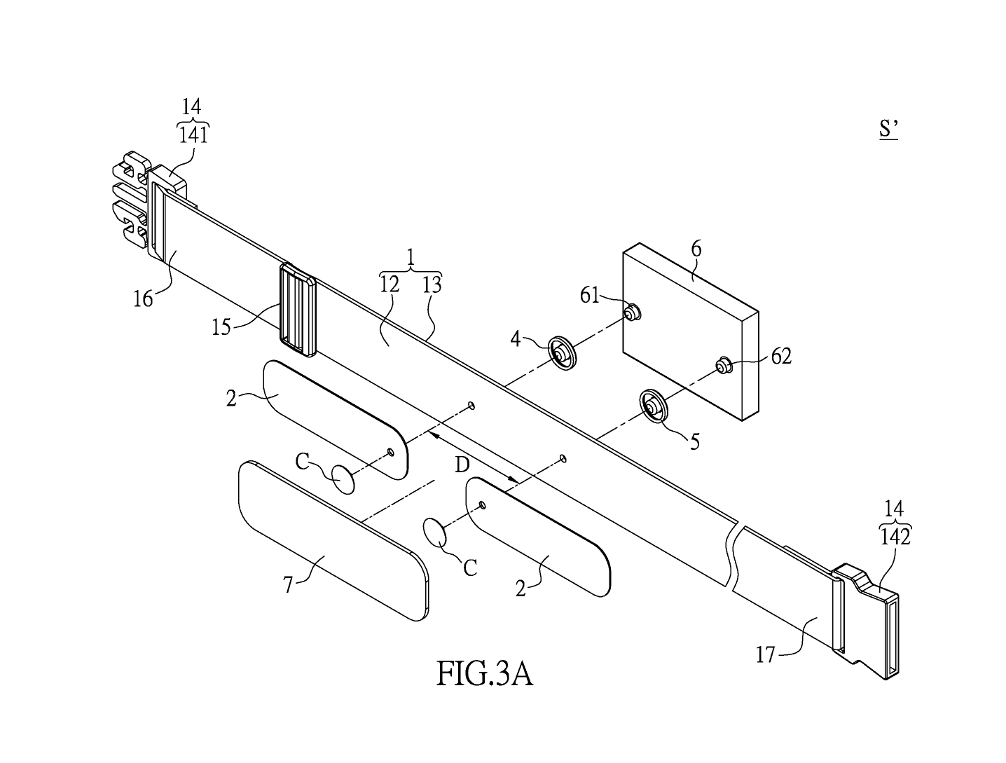

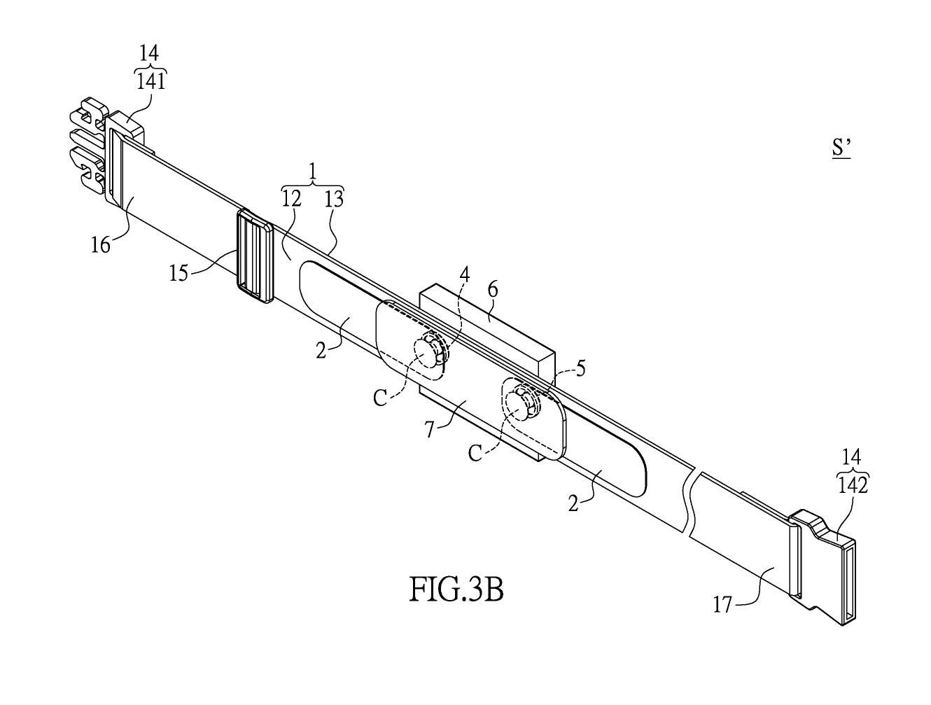

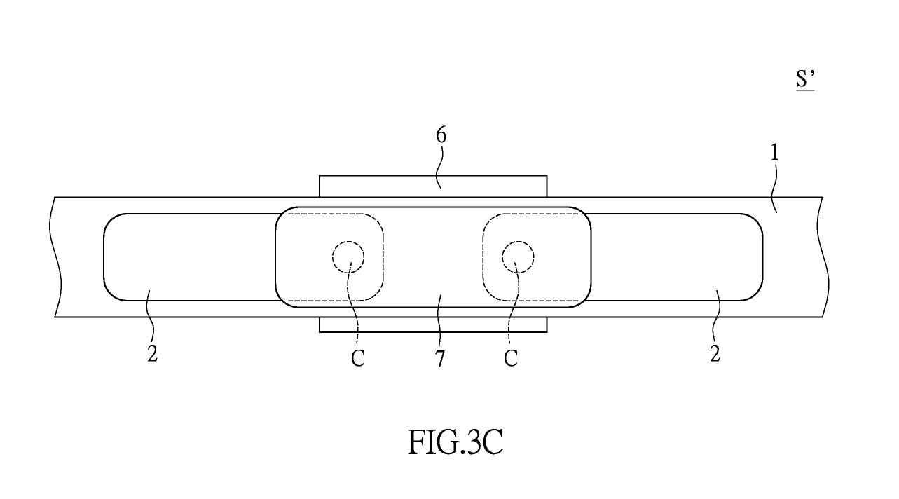

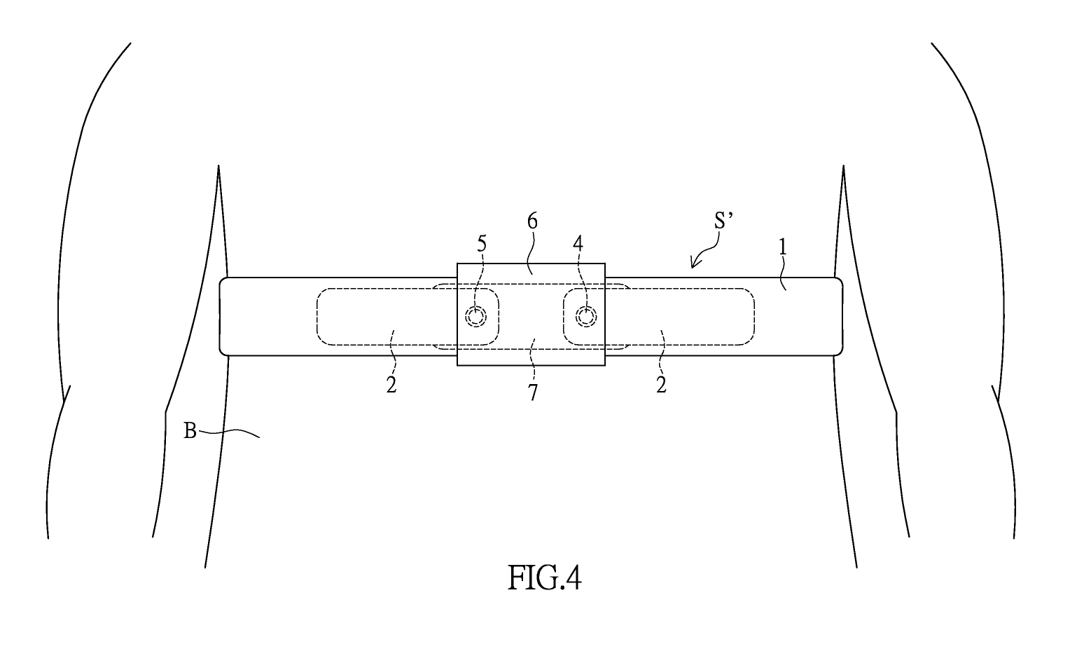

Please refer to FIG. 3A to FIG. 3C. FIG. 3A shows an exploded view of the physiological signal monitoring belt according to the second embodiment of the present disclosure. FIG. 3B shows a perspective view of the physiological signal monitoring belt according to the second embodiment of the present disclosure. FIG. 3C shows a front view of the physiological signal monitoring belt according to the second embodiment of the present disclosure. As comparing FIG. 3A and FIG. 1 show that one of the differences between the second embodiment and the first embodiment is: the physiological signal monitoring belt S of the embodiment can be used in conjunction with a signal transceiver 6.

The physiological signal monitoring belt S′ provided by the second embodiment includes a belt-shaped unit 1, two electrode units 2, a first engaging element 4, and a second engaging element 5. The belt-shaped unit 1 has a first surface 12 and a second surface 13. As a specific example, the first surface 12 of the belt-shaped unit 1 is configured to be in touch with the skin of the living body to be monitored, and the two electrode units 2 both are disposed on the first surface 12 of the belt-shaped unit 1 for monitoring the physiological information of the living body. To put it concretely, the two electrode units 2 are arranged correspondingly to each other and spaced apart from each other with a determined distance D. In the second embodiment of the instant disclosure, the first engaging element 4 and the second engaging element 5 are spaced apart from each other with a distance, which may be equal to the determined distance D.

Please refer concurrently to FIG. 2A and FIG. 2B. Specifically, each of the electrode units 2 may include a base layer 21, an intermediate layer 22, and a conductive layer 23. In other words, each of the electrode unit 2 can be formed with a multi-layered composite material. The intermediate layer 22 of each of the electrode units 2 has a first surface 221 and a second surface 222, which can be the lower surface and the upper surface of the intermediate layer 22. The base layer 21 of each of the electrode units 2 can be disposed on the first surface 221 of the corresponding intermediate layer 22, and the conductive layer 23 of each of the electrode units 2 can be disposed on the second surface 222 of the corresponding intermediate layer 22. Therefore, each of the electrode units 2 can be disposed on the first surface 12 of the belt-shaped unit 1 through the corresponding base layer 21. Each of the electrode units 2 of the second embodiment of the present disclosure is similar to the electrode unit 2 of the first embodiment, and the features which can be similar to those of the first embodiment are not further described. For example, the physiological signal monitoring belt S′ of the second embodiment can utilizes the electrode unit 2′ provided in the first embodiment. It is worth noting that, each of the electrode units 2 can be fixedly attached to the belt-shaped unit 1 through a layering unit 8, which is similar to that of the first embodiment, peripherally arranged on the electrode unit 2, and the present disclosure is not limited thereto.

The first engaging element 4 is electrically connected to one of the two electrode units 2, and the second engaging element 5 is electrically connected to the other of the two electrode units 2. The first engaging element 4 and the second engaging element 5 are spaced apart from each other with a determined distance D. For example, the physiological signal monitoring belt S′ of the second embodiment can have two fastening elements C disposed thereon. The two fastening elements C are arranged correspondingly to the first engaging element 4 and the second engaging element 5 respectively, and the two fastening elements C are arranged correspondingly to each other. To put it concretely, the first engaging element 4 and one of the two fastening elements C are configured to be fastened together, such that one of the electrode units 2 is positioned there between. The second engaging element 5 and the other of the two fastening elements C are configured to be fastened together, such that the other of the electrode units 2 is positioned there between. It is worth mentioning that, the first engaging element 4 and the second engaging element 5 are formed of electrically conductive materials (such as rivet buttons). After fastened with the fastening elements C, the first engaging element 4 and the second engaging element 5 must have electrically conductive properties.

The physiological signal monitoring belt S′ of the second embodiment can be used in conjunction with a signal transceiver 6. Specifically, the signal transceiver 6 has a first signal transmitting terminal 61, which is corresponding to the first engaging element 4, and a second transmitting terminal 62, which is corresponding to the second engaging element 5. As a specific example, the structure of the end of the first engaging element 4 that is apart from the corresponding electrode unit 2 is formed with an aperture (not shown in the Figures), whereby the first signal transmitting terminal 61 of the signal transceiver 6 and the first engaging element 4 can be fastened together through the aperture. Similarly, the structure of the end of the second engaging element 5 that is apart from the corresponding electrode unit 2 is formed with an aperture (not shown in the Figures), whereby the second signal transmitting terminal 62 of the signal transceiver 6 and the second engaging element 5 can be fastened together through the aperture. Accordingly, the first signal transmitting terminal 61 is electrically connected to the first engaging element 4, and the second signal transmitting terminal 62 is electrically connected to the second engaging element 5, whereby the physiological information of the living body observed by the electrode units 2 can be transferred to the signal transceiver 6 and then transmitted to an external electronic device (not shown in the Figures) by the signal transceiver 6 to be read, analyzed, or displayed. For example, the physiological signal monitoring belt S′ can also be worn around the neck, the arm, or the other part of the living body to observe the physiological information of the living body such as ECG, EEG, body temperature, pulse rate, and the like.

To prevent the signal transceiver 6 from malfunction due to the sweating water, the physiological signal monitoring belt S′ may further include an isolating element 7 positioned between the two electrode units 2 for isolating water. The isolating element 7 covers portions of the two electrode units 2 and a portion of the belt-shaped unit 1 that is positioned between the two electrode units 2. As a specific example, the isolating element 7 can be formed with an electrically insulating material, and the isolating element 7 can be attached to the first surface 12 of the belt-shaped unit 1 by gluing, sewing set, ultrasonic bonding or thermo-compression bonding. Moreover, the two fastening elements C can be covered by the isolating element 7, which prevents the fastening elements C from being in direct touch with the skin of the living body, thus the discomfort due to the fastening elements C can be avoided. Furthermore, since the noticeable fastening elements C are covered by the isolating element 7, a preferred aesthetic effect is achieved. It is worth noting that, the isolating element 7 also facilitates the fastening elements C to be fixedly disposed on the belt-shaped unit 1, meanwhile, the fastening elements C are kept from being in direct touch with living body, thus the transferring of the signals to the signal transceiver 6 though the fastening elements C will not be affected.

The belt-shaped unit 1 of the second embodiment further has a connecting element 14 and an adjustment structure 15 positioned on two end portions of the belt-shaped unit 1. Specifically, the connecting element 14 includes a first connecting portion 141 and a second connecting portion 142. The belt-shaped unit 1 has a first end portion 16 and a second end portion 17. The first connecting portion 141 is disposed on the first end portion 16 of the belt-shaped unit 1, and the second connecting portion 142 is disposed on the second end portion 17 of the belt-shaped unit 1. The adjustment structure 15 is disposed between the first end portion 16 and the second end portion 17. The adjustment structure 15 enables the user to adjust length of the belt-shaped unit 1 and the distance between the first connecting portion 141 and the second connecting portion 142 according to needs, thus the physiological signal monitoring belt S′ can be adjusted to a desired tightness when worn on the living body. As a specific example, the first connecting portion 141 and the second connecting portion 142 may be a pair of magnets assembly, rivet button assembly, or other fastening assembly that can be detachably engaged to each other. Conventional connecting means well known to those skilled in the art may be employed to form the first connecting portion 141 and the second connecting portion 142 and the instant disclosure is not limited thereto.

Please refer to FIG. 4, which shows a perspective view of the physiological signal monitoring belt in use according to the second embodiment of the present disclosure. The physiological signal monitoring belt S′ of the second embodiment can be disposed on the living body to be monitored. To put it concretely, the living body can be a human body B, and the present disclosure is not limited thereto. For example, the physiological signal monitoring belt S′ according to the instant embodiment can be used on any living body having a heartbeat. The first surface 12 of the belt-shaped unit 1 is arranged peripherally around the human body B and attached thereto, and the second surface 13 of the belt-shaped unit 1 is exposed. The first connecting portion 141 and the second connecting portion 142 respectively disposed on the first end portion 16 and the second end portion 17 of the belt-shaped unit 1 can be fastened to each other, such that the belt-shaped unit 1 is worn around the human body B. To avoid the discomfort due to long-time direct contact between the belt-shaped unit 1 and the human body B, the belt-shaped unit 1 of the second embodiment of the present disclosure can be configured to be moisture absorbent and sweat repellent. It is worth mentioning that, as a specific example of the second embodiment of the present disclosure, the physiological signal monitoring belt S′ can be disposed on clothing, such as a tank top or a sport vest, by weaving, stitching, or the like. Accordingly, the physiological signal monitoring belt S′ can be kept in touch with the human by the clothing that fits the human body B without using the connecting element 14 or adjustment structure 15, which are configured for facilitating the belt-shaped unit 1 to be tied on the human body B.

In accordance with the second embodiment, the physiological signal monitoring belt S′ provided by the present disclosure utilizes the electrode unit 2 that is formed with a multi-layered composite material to improve the monitoring effect and the monitoring accuracy thereof. Moreover, the physiological signal monitoring belt S′ utilizes the electrical connection between the electrode unit 2 and the signal transceiver 6 to transmit the physiological signal of the living body to an external electronic device through the signal transceiver 6, whereby the physiological information of the living body can be read, analyzed, or displayed.

### Effect of the Embodiments

To sum up, in accordance with the second embodiment, the physiological signal monitoring belt S, S′ utilizes the electrode unit 2, 2′ that is formed with a multi-layered composite material to improve the monitoring effect and the monitoring accuracy thereof. Especially, the electrode unit 2, 2′ can utilize a multi-layered structure having a base layer 21, an intermediate layer 22, and a conductive layer 23, to improve the signal monitoring accuracy for various physiological information. Meanwhile, the physiological signal monitoring belt S, S′ utilizes the micro pores 231 disposed on the conductive layer 23 to transfer the moisture absorbed by the intermediate layer 22 to the conductive layer 23, thus to increase the conductivity of the electrode unit 2′ that is in touch with the skin of the living body. Moreover, the physiological signal monitoring belt S, S′ utilizes the electrical connection between the electrode unit 2, 2′ and the signal transceiver 6 to transmit the physiological signal of the living body to an external electronic device through the signal transceiver 6, whereby the physiological information of the living body can be read, analyzed, or displayed.

The descriptions illustrated supra set forth simply the preferred embodiments of the present disclosure; however, the characteristics of the present disclosure are by no means restricted thereto. All changes, alterations, or modifications conveniently considered by those skilled in the art are deemed to be encompassed within the scope of the present disclosure delineated by the following claims.

What is claimed is:

1. A physiological signal monitoring belt, comprising:
a belt-shaped unit; and
an electrode unit disposed on a surface of the belt-shaped unit, wherein the electrode unit includes a base layer, an intermediate layer, and a conductive layer, the intermediate layer has a first surface and a second surface, the base layer is disposed on the first surface, and the conductive layer is disposed on the second surface;
wherein the electrode unit is disposed on the surface of the belt-shaped unit through the base layer.

2. The physiological signal monitoring belt of claim 1, wherein the conductive layer has a plurality of micro pores.

3. The physiological signal monitoring belt of claim 1, wherein the intermediate layer is formed with a non-woven cloth, a water absorbent sponge or a water absorbent colloid.

4. The physiological signal monitoring belt of claim 1, wherein the conductive layer is formed with a rubber conductive layer or a conductive fabric.

5. The physiological signal monitoring belt of claim 1, wherein the intermediate layer is water absorbent.

6. The physiological signal monitoring belt of claim 1, wherein the base layer is formed with an adhesive colloid layer.

7. A physiological signal monitoring belt, comprising:
a belt-shaped unit having a first surface and a second surface;
two electrode units disposed on the first surface of the belt-shaped unit, wherein the two electrode units are arranged correspondingly to each other and each include a base layer, an intermediate layer, and a conductive layer, the intermediate layer has a first surface and a second surface, the base layer is disposed on the first surface of the intermediate layer, and the conductive layer is disposed on the second surface of the intermediate layer;
a first engaging element electrically connected to one of the two electrode units; and
a second engaging element electrically connected to the other of the two electrode units;
wherein each of the two electrode units is disposed on the first surface of the belt-shaped unit through the corresponding base unit.

8. The physiological signal monitoring belt of claim 7, further comprising: a signal transceiver having a first signal transmitting terminal corresponding to the first engaging element and a second transmitting terminal corresponding to the second engaging element, wherein the first signal transmitting terminal is electrically connected to the first engaging element, and the second signal transmitting terminal is electrically connected to the second engaging element.

9. The physiological signal monitoring belt of claim 7, wherein the two electrode units each have a plurality of micro pores.

10. The physiological signal monitoring belt of claim 7, further comprising: an isolating element positioned between the two electrode units and covering portions of the two electrode units.
